(19) United States
(12) Patent Application Publication
(10) Pub. No.: US 2022/0372500 A1
(43) Pub. Date: Nov. 24, 2022

(54) ATTENUATED NOCARDIA SERIOLAE AND CONSTRUCTION METHOD AND USE THEREOF

(71) Applicants: GUANGDONG OCEAN UNIVERSITY, Zhanjiang, Guangdong (CN); SHENZHEN INSTITUTE OF GUANGDONG OCEAN UNIVERSITY, Shenzhen, Guangdong (CN)

(72) Inventors: Liqun XIA, Zhanjiang, Guangdong (CN); Wenji WANG, Zhanjiang, Guangdong (CN); Yishan LU, Zhanjiang, Guangdong (CN); Zhen GAN, Zhanjiang, Guangdong (CN); Suying HOU, Zhanjiang, Guangdong (CN)

(21) Appl. No.: 17/428,791

(22) PCT Filed: Feb. 5, 2021
(86) PCT No.: PCT/CN2021/075555

§ 371 Date: Aug. 5, 2021

(30) Foreign Application Priority Data
Dec. 7, 2020 (CN) 202011430248.3

(51) Int. Cl. C12N 15/76; C12N 9/52; C12N 1/20; A61K 39/05; A61P 31/04

(52) U.S. Cl. CPC C12N 15/76; C12N 9/52; C12N 1/20; A61K 39/05; A61P 31/04; A61K 2039/522

(57) ABSTRACT

The present disclosure provides an attenuated Nocardia seriolae and its construction method and use thereof, which belongs to the technical field of genetic engineering. In the present disclosure, part or all of the glutamate endopeptidase homologue (GluNS) gene sequence of wild-type Nocardia seriolae is knocked out by means of genetic engineering to construct an attenuated strain with the characterized of good protection to the host. The attenuated strain may not only effectively reduce the pathogenicity of the bacteria, but also retain good immunogenicity. At the same time, the attenuated Nocardia seriolae constructed in the present disclosure may also have the characteristics of high genetic stability and may be used as a vaccine candidate strain for the preparation of a vaccine or other biological products for preventing and treating Nocardiosis.

### CROSS REFERENCE TO RELATED APPLICATION

This patent application is a U.S. National Phase Application under 35 U.S.C. § 371 of International Patent Application No. PCT/CN2021/075555, filed Feb. 5, 2021, claims the benefit and priority of Chinese Patent Application No. 202011430248.3 entitled “Attenuated Nocardia seriolae and Construction Method and Use Thereof” filed on Dec. 7, 2020, the disclosure of which is incorporated by reference herein in its entirety as part of the present application.

### TECHNICAL FIELD

The present disclosure belongs to the technical field of genetic engineering, and specifically relates to an attenuated Nocardia seriolae, its construction method and use thereof.

### BACKGROUND ART

In recent years, fish nocardiosis has become a serious disease in the aquaculture industry. When aquatic animals are weak and immunocompromised, pathogens can be infected through gills, feed or wounds. Diseased fish are manifested by enlarged abdomen and dense granulomas in internal organs. The disease characterized by high infection rate and mortality rate, causing huge losses to the aquaculture industry due to the lack of effective prevention and control measures at present. Recently, Nocardia seriolae (N. seriolae) has become the main causative agent of nocardiosis, and more than 39 kinds of cultured marine and freshwater fish species suffer from it.

Relevant studies have shown that the main pathogens of fish nocardiosis is N. seriolae. N. seriolae belongs to Nocardia genus, Nocardiaceae family, Actinobacterales order, Actinobacteria class, Firmicutes phylum in Bacteria domain. N. seriolae grows slowly. The bacteria grows on a plate and presents yellow sandy or granular protrusions with folds on the surface. Under liquid culture conditions, bacteria tend to aggregate to form large particles^{[1]}. At present, the prevention and treatment of N. seriolae still relies on precautions. As an active bacteria, the attenuated strain of bacteria does not harm to the host while maintaining the original immunogenicity. Therefore, the attenuated strain has attracted much attention. Related studies have been exploring the construction of attenuated strains of N. seriolae. However, the protective effect of related attenuated strains is still not desirable and lacks immunogenicity.

### SUMMARY

In view of above aspects, the purpose of the present disclosure is to provide an attenuated Nocardia seriolae (N. seriolae), its construction method and application thereof. The constructed N. seriolae has the characteristics of low pathogenicity and still retains good immunogenicity. It may be used as a candidate strain in the preparation of vaccines or other biological products.

The present disclosure provides an attenuated N. seriolae, and the GluNS gene is deleted in the attenuated N. seriolae.

In some embodiments, the amino acid sequence of the protein encoded by the GluNS gene is set forth in SEQ ID NO: 12 or a sequence that has a homology or coverage of 30% to 99% with SEQ ID NO: 12.

In some embodiments, the attenuated N. seriolae may be deposited with an accession number of GDMCC No: 61258.

The present disclosure provides a method for constructing an attenuated N. seriolae, which may include the following steps:

1) inserting a homologous recombinant arm of the GluNS gene into a vector to construct a recombinant vector;

2) transforming the recombinant vector into N. seriolae competent cells to obtain recombinant cells;

3) culturing and screening the recombinant cells to obtain an attenuated N. seriolae.

In some embodiments, the homologous recombinant arm of the GluNS gene in step 1) comprises an upstream fragment and a downstream fragment of the GluNS gene.

In some embodiments, the length of the upstream fragment or downstream fragment of the GluNS gene may independently be 5 bp to 2000 bp.

In some embodiments, the upstream fragment of the GluNS gene may have a 389 bp fragment upstream of the GluNS gene as the upstream homology arm, the downstream fragment may have a 407 bp fragment downstream of the GluNS gene as the downstream homology arm, and the homologous recombinant arm of the GluNS gene may be formed in a manner of ligating the upstream homology arm and the downstream homology arm.

In some embodiments, the nucleotide sequence of the homologous recombinant arm of the GluNS gene in step 1) is set forth in SEQ ID NO:1.

In some embodiments, the vector in step 1) may include pRE112;

the multiple cloning site where the homologous recombinant arm of the GluNS gene is inserted into pRE112 may be Sac I/Kpn I.

In some embodiments, the transformation in step 2) may include electroporation;

the parameters of the electroporation may preferably be as follows: voltage 180-220 V, pulse interval time 800-1200 ms, pulse duration 80-120 μs, and square wave 25-35.

In some embodiments, the culture solution in step 3) may be a BHI solution containing 0.3 mM sucrose preheated at 26-30° C., the culture temperature may be 26-30° C., and the culture time may be 10-12 h.

In some embodiments, the screening may include detecting whether the bacteria contains the knockout plasmid pRE112-ΔGluNS by a PCR amplification method;

the primer pairs for PCR amplification are 112-F1/112-R1;

the nucleotide sequence of 112-F1 is set forth in SEQ ID NO: 2;

the nucleotide sequence of 112-R1 is set forth in SEQ ID NO: 3;

Clones in which the GluNS gene fragment is shown to be deleted via a PCR amplification product sequencing, and the bacteria contains a knockout plasmid pRE112-ΔGluNS are positive clones.

In some embodiments, after the screening, the method may further include an identification of the attenuated N. seriolae;

the identification includes identifying whether the GluNS gene is deleted by a PCR amplification method;

the primer pairs for PCR amplification are GluNS-F1/GluNS-R1 and GluNS-F2/GluNS-R2;

the nucleotide sequence of GluNS-F1 is set forth in SEQ ID NO: 10;

the nucleotide sequence of GluNS-R1 is set forth in SEQ ID NO: 11;

the nucleotide sequence of GluNS-F2 is set forth in SEQ ID NO: 14;

the nucleotide sequence of GluNS-R2 is set forth in SEQ ID NO: 15;

The target band could not be amplified from GluNS gene deletion strains by using the GluNS-F1/GluNS-R1 primer pair; only a 796 bp upstream and downstream homologous band could be amplified from GluNS gene deletion strains by using the GluNS-F2/GluNS-R2 primer pair.

The present disclosure provides an application of the attenuated N. seriolae in the preparation of vaccines or related biological products containing the attenuated N. seriolae.

The present disclosure provides an attenuated N. seriolae. An attenuated strain, with good protection to the host, is constructed by knocking out the glutamate endopeptidase homologue (GluNS) gene in the wild-type N. seriolae by genetic engineering. The attenuated strain may not only effectively reduce the pathogenicity of the bacteria, but also retain good immunogenicity. Experiments show that the median lethal concentration of the wild strain is 4.74×10⁵ CFU/mL, and the median lethal concentration of the attenuated strain is 3.41×10⁶ CFU/mL, which is one order of magnitude lower than that of the wild strain, indicating that the virulence of this strain decreased significantly. The results of viable bacterial challenge experiments show that the relative immune protection rate of the attenuated strain reaches 93.38%, indicating that the attenuated strain may be an attenuated strain with good protection to the host (the strain is deposited with an accession number of GDMCC No: 61258).

At the same time, the attenuated N. seriolae constructed in the present disclosure may also has the characteristics of high genetic stability. Experiments show that the GluNS gene fragment could not be detected in the GluNS deletion strain of N. seriolae ZJ0503-6296 which has been continuously transmitted to 30 generations, indicating that the GluNS gene deletion strain can be inherited stably.

### BRIEF DESCRIPTION OF THE DRAWINGS

FIG. 1 is a plasmid map of pRE112;

FIG. 2 is a map of recombinant plasmids constructed in the present disclosure;

FIG. 3 is an electrophoresis map showing the first screening of the GluNS gene deletion strain Nocardia seriolae (N. seriolae) ZJ0503-6296; M: DNA marker 2000, 1-2: experimental group, 3: ZJ0503, 4: pRE112;

FIG. 4 is an electrophoresis pattern showing the second screening of the GluNS gene deletion strain N. seriolae ZJ0503-6296; M: DNA marker 2000, 1: N. seriolae ZJ0503-6296, 2: ZJ0503;

FIG. 5 shows the identification of genetic stability of the GluNS gene deletion strain N. seriolae ZJ0503-6296; M: DNA marker 10000, 1: N. seriolae ZJ0503-6296, 2-4: ZJ0503;

FIG. 6 is a schematic diagram showing the construction method of the knockout plasmid pRE112-ΔGluNS;

FIG. 7 is the construction principle of the GluNS gene deletion strain N. seriolae ZJ0503-6296.

### BIOLOGICAL MATERIAL PRESERVATION INFORMATION

Nocardia seriolae (Nocardia sp.) ZJ0503-6296, is deposited in Guangdong Microbial Culture Collection Center, which can be referred to as GDMCC and its address is Guangzhou, Guangdong Institute of Microbiology, 5th Floor, Building 59, No. 100 Xianlie Middle Road. The deposition date was Oct. 29, 2020, and the accession number is GDMCC No: 61258.

### DETAILED DESCRIPTION OF THE EMBODIMENTS

The present disclosure provides an attenuated Nocardia seriolae (N. seriolae), and the GluNS gene is deleted in the attenuated N. seriolae.

In the present disclosure, there are no special restrictions on the GluNS gene sequence, as long as it can achieve the object of attenuation by deleting all the nucleotide sequence (SEQ ID NO: 13, gtgctcgtcgccggacccctggcggcgagcgcacacgcggaacccgcgacccccgatctgcccgcccagctgatcgcgg ccatcacccgcgacctcaagatctccccgcaggactacctggcccgggccgacaccgcacagaaggtggccaccttcgcc accaccgcgcagcggcagttcccgcaggtcttcggcggcgcctggctggacgagaccggcaaggccgtcgtggcgctgg ccccgggcgagggcgtggacaaggcccgcaaggccgtgcaggacgccggtttcaccgccaaggacgtgagcaagagcg agaccacgctgcgcggcgagaagaacgccttccagcagtggctcaaggatcagcccgagtccgtggcgaaggccatccg gggcgtggccatcgacaccttgaacaacagcatcgcggtgcgggtggacaagcccgatctgccgctgccgggcttcgtgga cccggcccgcgtcatcgtcatgaccgcaccgccggtcggcggcgagggccagaacgtgccgcaggccaccgagatcgc gggcgcgggcccgcgcgccatcgccgccggcgaggcctacgcctcggtcgccggccgcatgtcgctgcgctgctcgctc ggcttcaacggcaccgacggcaacggcaatgtcgtcaatatcaccgcgggccactgcaacccgaacatcgcggccaccgg cggcgcgaactcgccgagcgtgtaccagctcatcggtgacagccgcggtcccgaggtcggccagttccagaagtcggtgct gggcaacgaggactactcgatcgtcggcatcaacgaccagttccgcgacgccttctccaacccgttcgtaaccgtcccgggc tcggcctcgatcgccgtcaccggcgtggccgtgccggtggtcggcgcgccggtctgcaagtcgggtgcgcgcaccggcttc agctgcggcgtggtgaacgccgtggaccagaccgtccaggtcggcgaccgcctgctgacccagtccttctccgccaatatct gtgccctgcccggtgattcgggtggcccgctggtgaccggcacgctggccctgggcatcgccagcgcatcctcggtcgccg actacccgatctgcgagatcccgaacctgctcggcctgatcaccggcaacaccccgcagctgttcgcgcagccggtgagca ccgtgctctccgacaacccggggctgcgggtccgcaccacgtaa) or part of the nucleotide sequence (ORF6296) of the GluNS gene known in the art. Therefore, the deletion of all or part of the nucleotide sequence of the GluNS gene and the encoded amino acid sequence are within the scope of the present disclosure.

In the present disclosure, the amino acid of the protein encoded by the GluNS gene is preferably set forth in SEQ ID NO: 12 (VLVAGPLAASAHAEPATPDLPAQLIAAITRDLKISPQDYLARADTAQKVATFATT AQRQFPQVFGGAWLDETGKAVVALAPGEGVDKARKAVQDAGFTAKDVSKSET TLRGEKNAFQQWLKDQPESVAKAIRGVAIDTLNNSIAVRVDKPDLPLPGFVDPAR VIVMTAPPVGGEGQNVPQATEIAGAGPRAIAAGEAYASVAGRMSLRCSLGFNGT DGNGNVVNITAGHCNPNIAATGGANSPSVYQLIGDSRGPEVGQFQKSVLGNED YSIVGINDQFRDAFSNPFVTVPGSASIAVTGVAVPVVGAPVCKSGARTGFSCGVV NAVDQTVQVGDRLLTQSFSANICALPGDSGGPLVTGTLALGIASASSVADYPICEI PNLLGLITGNTPQLFAQPVSTVLSDNPGLRVRTT). The protein encoded by the deleted GluNS gene in the present disclosure may also include a sequence that has a homology or coverage of 30% to 99% with SEQ ID NO:12.

The attenuated N. seriolae is preferably deposited with an accession number of GDMCC No: 61258.

The present disclosure further provides a method for constructing an attenuated N. seriolae, which includes the following steps:

1) inserting the homologous recombinant arm of the GluNS gene into a vector to construct a recombinant vector;

2) transforming the recombinant vector into N. seriolae competent cells to obtain recombinant cells;

3) culturing and screening the recombinant cells to obtain an attenuated N. seriolae.

The present disclosure provides a method for constructing the attenuated N. seriolae, the principle of which is that about 500 bp fragments upstream and downstream of the target gene are cloned as the homology arms, and the homology arms are ligated together by overlapping PCR method to construct on the deletion vector pRE112. The deletion vector is introduced into N. seriolae, and the homologous recombinant arm fragment on the deletion vector is replaced with the homologous arm and the target gene fragment in the bacteria by the homologous recombination method, so that the target gene in N. seriolae may be deleted, and only contains the recombined homologous arm fragment, and the attenuated N. seriolae is thereby constructed (FIG. 7).

In the present disclosure, the homologous recombinant arm of the GluNS gene is inserted into a vector to construct a recombinant vector.

In the present disclosure, the homologous recombinant arm of the GluNS gene preferably comprises an upstream fragment and a downstream fragment of the GluNS gene. There are no special restrictions on the length of the upstream fragment and the downstream fragment. The length of the upstream fragment and the downstream fragment of the GluNS gene well known in the art can be employed, for example, 5 bp to 2000 bp. In an embodiment of the present disclosure, the upstream fragment of the GluNS gene has a 389 bp fragment upstream of the GluNS gene as the upstream homology arm, the downstream fragment has a 407 bp fragment downstream of the GluNS gene as the downstream homology arm, and the homologous recombinant arm of the GluNS gene is formed in a manner of ligating the upstream homology arm and the downstream homology arm. The nucleotide sequence of the homologous recombinant arm of the GluNS gene is preferably set forth in SEQ ID NO: 1 (GCCTGCACCCTGGGCGGTGACAGCGGCGGCGCGATCGTGTCGGGCACGCTG GCGCTGGGCATCACCAGCGGTTCGAACGCGGCCGACGCGCCGAACTGCAAC GAGGCCAACACCGCGCTGGCGCAGTACGGTGGAACCGCGTCGCTGGGCATC CCGGTGCGCGCGATTCTCACCGAAATCGACGCCAATTCCGGTGGCGGCGTCG GCAGCGGAATTCAGGTGCGGACGCGGTCCAACGCCTGACGGCGAAGTGCTC CAGCACCCGACGGCAAAGCGGTCCAACGCCTGACGATGGCGACATCGGCGG TAAACGGACATCCTTCACATTGCCCCTGAATCCGACATGCCCGCTGTTTGCCC CGTGAATACCGAAACGTCACCTCGCAATAAGTAGGCATAACGGGCCCGACAC CATATAGTCGGGGCCATGCTCCTGCCACGTATGCGCTTCTCCATGCCTGCCGC GGGCCGACGCCCGCGGTCCCTTGTCCGTACTGCGGCGATCGCGGCGACCTCT TCACGGCACCCTCGAAAAGGCCGGTAGCTCAACAGCTACCGGCCTTTTGTCG TCCCGGTGGGGAAGAGATACCGCTGACCTCACTAATTCGGATGCAGCGCTGG AAGTTCGTGGCCCGCCTCGGTCTGGACTGACGGAGCGGGGGAGCGAAGCGG AGGAGCGGAGGAGGGAAGACCGAGGTAACAGGGCCACCAACCGCCGTAGC GCAGCGGAGGCAAATTAAACAGAGCATGCTGGGGATATGTGTTTGGTGTTGC TGGGTTGGCGAGCGCATCCGGAGTACCGCTTGATCGTGGCCGCCAACCGGGA CGAGTTCTTCACCCGCCCAACGGAATCGCTGCGCCGGTGGGACGAAGTGCC CGGGGTGCTGGCCGGGCGGGACCTGGGCGCGGCCGGACCGGTGCCCGGCAC CTGGCTCGGTGCGCTGCCCGATCATCGCCGCTTCGCGACGGTCACCAATGTG CGACTACCCGGCGAATTCCGCGCCGATGTGCGCTCGCGCGGCGCGCTGCTGC TGG). The nucleotide sequence of the upstream fragment of the GluNS gene is preferably set forth in SEQ ID NO: 4 (GCCTGCACCCTGGGCGGTGACAGCGGCGGCGCGATCGTGTCGGGCACGCTG GCGCTGGGCATCACCAGCGGTTCGAACGCGGCCGACGCGCCGAACTGCAAC GAGGCCAACACCGCGCTGGCGCAGTACGGTGGAACCGCGTCGCTGGGCATC CCGGTGCGCGCGATTCTCACCGAAATCGACGCCAATTCCGGTGGCGGCGTCG GCAGCGGAATTCAGGTGCGGACGCGGTCCAACGCCTGACGGCGAAGTGCTC CAGCACCCGACGGCAAAGCGGTCCAACGCCTGACGATGGCGACATCGGCGG TAAACGGACATCCTTCACATTGCCCCTGAATCCGACATGCCCGCTGTTTGCCC CGTGAATACCGAAACGTCACCTCGCAATAAGTAGGCATAACGGGCCCGACAC CATATAGTCGGGGCCATGCTCCTGCCACGTATGCGCTTCTCCATGCCTGCCGC GGGCCGACGCCCGCGGTCCCTTGTCCGTACTGCGGCGATCGCGGCGACCTCT). The primers for amplification of the upstream fragment of the GluNS gene may preferably be GluNS-UF and GluNS-UR. The nucleotide sequence of the GluNS-UF is preferably set forth in SEQ ID NO: 5 (CATATGGGGCATCACCAGCGGTTCG), and the nucleotide sequence of the GluNS-UR is preferably set forth in SEQ ID NO: 6 (TGCATCCGAAT TAGTGAGGTCAGAGGAGCATGGCCCCGACT). The nucleotide sequence of the downstream fragment of the GluNS gene is preferably set forth in SEQ ID NO: 7 (TCACGGCACCCTCGAAAAGGCCGGTAGCTCAACAGCTACCGGCCTTTTGTC GTCCCGGTGGGGAAGAGATACCGCTGACCTCACTAATTCGGATGCAGCGCTG GAAGTTCGTGGCCCGCCTCGGTCTGGACTGACGGAGCGGGGGAGCGAAGCG GAGGAGCGGAGGAGGGAAGACCGAGGTAACAGGGCCACCAACCGCCGTAG CGCAGCGGAGGCAAATTAAACAGAGCATGCTGGGGATATGTGTTTGGTGTTG CTGGGTTGGCGAGCGCATCCGGAGTACCGCTTGATCGTGGCCGCCAACCGGG ACGAGTTCTTCACCCGCCCAACGGAATCGCTGCGCCGGTGGGACGAAGTGC CCGGGGTGCTGGCCGGGCGGGACCTGGGCGCGGCCGGACCGGTGCCCGGCA CCTGGCTCGGTGCGCTGCCCGATCATCGCCGCTTCGCGACGGTCACCAATGT GCGACTACCCGGCGAATTCCGCGCCGATGTGCGCTCGCGCGGCGCGCTGCTG CTGG). The primers for amplification of the downstream fragment of the GluNS gene may preferably be GluNS-DF and GluNS-DR. The nucleotide sequence of GluNS-DF is preferably set forth in SEQ ID NO: 8 (AGTCGGGGCCATGCTCCTCTGACCTCACTAATTCGGATGCA), and the nucleotide sequence of GluNS-DR is preferably set forth in SEQ ID NO: 9 (GAGCTCAATTCGCCGGGTAGTCGC). Overlapping PCR products are obtained by PCR amplification, with GluNS-UF and GluNS-DR as primers, and upstream and downstream fragment of the GluNS gene as templates. The PCR amplification program is: 94° C., 5 min, 15 cycles: 94° C., 30 s, 58° C., 30 s, 72° C., 1 min, and 72° C., 5 min. The reaction system is: upstream fragment product of the GluNS gene 2 μL, downstream fragment product of the GluNS gene 2 μL, GluNS-UF and GluNS-DR each 1 μL, rTaq enzyme 25 μL, and sterile water 19 μL.

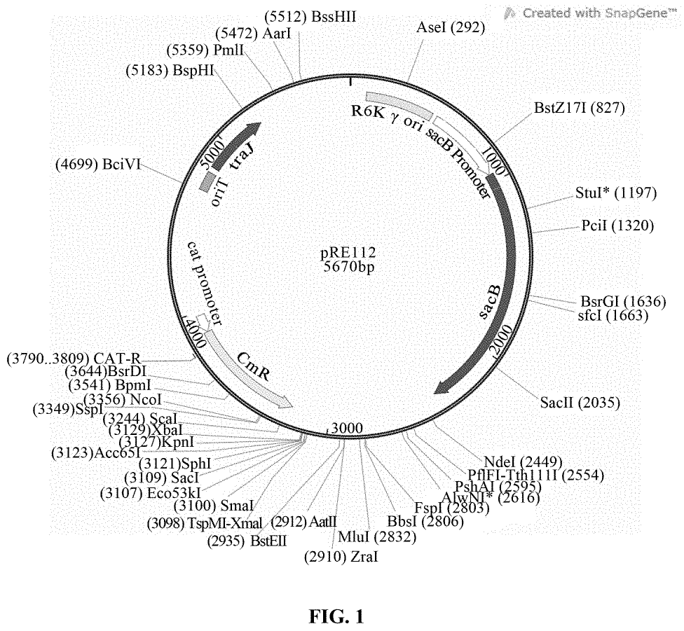

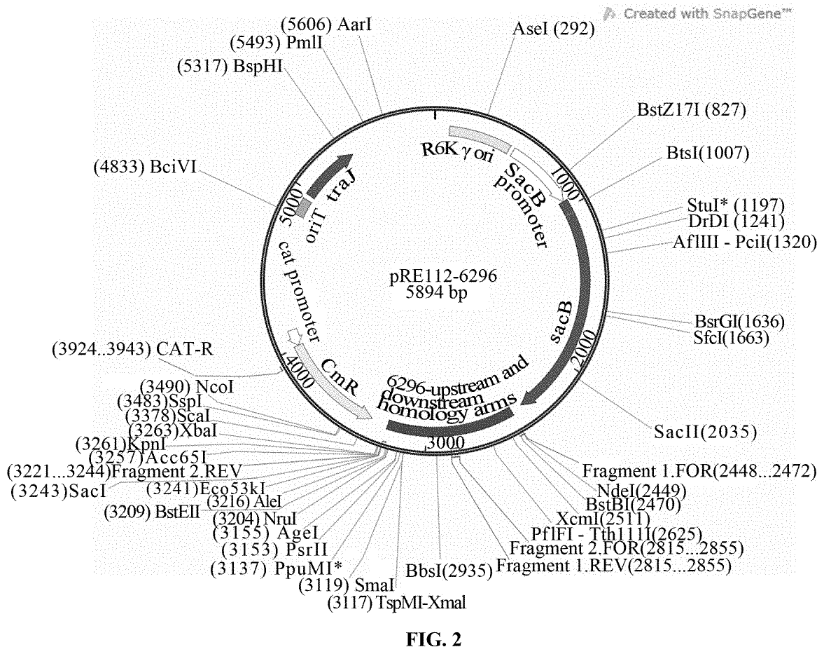

In the present disclosure, the vector preferably includes pRE112, as shown in FIG. 1. the multiple cloning site where the homologous recombinant arm of GluNS gene is inserted into pRE112 is preferably Sac I/Kpn I. The method for inserting the homologous recombinant arm of the GluNS gene into the vector preferably includes enzyme digestion and ligation. The enzyme digestion includes digestion of the homologous recombinant arm of the GluNS gene and the vector separately. In the present disclosure, there are no special limitations on the enzyme digestion method, and a enzyme digestion method well known in the art will do. In the present disclosure, there are no special limitations on the ligation method, and a ligation method well known in the art will do, for example using T4 ligase for ligation. In the present disclosure, there are no special limitations on the ligation method of the T4 ligase, and a ligation scheme of the T4 ligase well known in the art will do. After the ligation, the technology scheme in the present disclosure further preferably includes an identification; the identification preferably adopts a PCR amplification method or a sequencing method. The constructed recombinant vector is set forth in in FIG. 2.

After obtaining the recombinant vector, the recombinant vector is transformed into N. seriolae competent cells to obtain recombinant cells.

In the present disclosure, a method known in the art for preparing competent cells is employed in the preparation of competent cells of N. seriolae. There are no special limitations on the source of N. seriolae, and N. seriolae well known in the art may be used. In order to illustrate the method for constructing the attenuated strain, in an embodiment of the present disclosure, the wild strain of N. seriolae ZJ0503 is used as the material for implementation.

In the present disclosure, there are no special limitations on the transformation method, and in an embodiment of the present disclosure, the transformation preferably includes electroporation; the parameters of the electroporation are preferably as follows: voltage 180-220 V, pulse interval time 800-1200 ms, pulse duration 80-120 μs, and square wave 25-35.

After obtaining the recombinant cells, the recombinant cells is cultured and screened to obtain an attenuated N. seriolae.

In the present disclosure, the culture solution is preferably a BHI solution containing 0.3 mM sucrose preheated at 26-30° C., the culture temperature is preferably 26-30° C., and the culture time is preferably 10-12 h. The screening preferably uses antibiotics corresponding to the resistance gene carried in the vector for screening. In an embodiment of the present disclosure, a BHI plate containing 25 mg/mL chloramphenicol resistance is preferably used for screening.

In the present disclosure, after the screening, the method preferably further include an identification of the attenuated N. seriolae. The identification preferably includes identifying whether the GluNS gene is deleted by PCR amplification method. The primer pairs for PCR amplification are preferably GluNS-F1/GluNS-R1 and GluNS-F2/GluNS-R2. The nucleotide sequence of GluNS-F1 is preferably set forth in SEQ ID NO: 10 (CCGCCAAGGACGTGAG). The nucleotide sequence of GluNS-R1 is preferably set forth in SEQ ID NO: 11 (CTGTCACCGATGAGCTGGTA). The nucleotide sequence of GluNS-F2 is preferably set forth in SEQ ID NO: 14 (GGGCATCACCAGCGGTTCG). The nucleotide sequence of GluNS-R2 is preferably set forth in SEQ ID NO: 15 (AATTCGCCGGGTAGTCGC). The PCR amplification reaction program is: 94° C., 5 min, 30 cycles: 94° C., 30 s, (55° C. (GluNS-F2/GluNS-R2), 58° C. (GluNS-F1/GluNS-R1), 30 s, 72° C., 1 min, and 72° C., 5 min.

The present disclosure provides an attenuated N. seriolae obtained by the construction method, and the GluNS gene is deleted in the genome of the attenuated N. seriolae. The median lethal concentration of the wild strain is 4.74×10⁵ CFU/mL, and the median lethal concentration of the attenuated strain is 3.41×10⁶ CFU/mL. The median lethal concentration of the attenuated strain is one order of magnitude lower than that of the wild strain, indicating that the virulence of this strain decreased significantly. The relative immune protection rate of the attenuated N. seriolae is 93.38%, and the attenuated N. seriolae has the characteristics of high immunogenicity. At the same time, the attenuated N. seriolae constructed in the present disclosure is directly knocked out from the N. seriolae genome. The attenuated N. seriolae does not contain resistant plasmid, which is in line with biological safety, and the growth of N. seriolae would not be affected by the loss of the plasmid.

In the present disclosure, in order to determine the genetic stability of the attenuated N. seriolae, the genetic stability of the attenuated N. seriolae is detected when it is passed for 30 generations. The method comprises amplifying by using primers GluNS-F1 (SEQ ID NO: 10) and GluNS-R1 (SEQ ID NO: 11), GluNS-F2 (SEQ ID NO: 14) and GluNS-R2 (SEQ ID NO: 15) to screen GluNS-deleted strains.

Based on the reduced virulence of the attenuated N. seriolae and the maintenance of ideal immunogenicity, the present disclosure provides applications of attenuated N. seriolae in preparation of vaccines or related biological products containing the attenuated N. seriolae. In the present disclosure, there are no special limitations on the preparation method of the vaccine, and a method for preparing a vaccine well known in the art will do. The prepared vaccines are used for preventing and treating fish nocardiosis caused by N. seriolae, and have high application values. In the present disclosure, there are no specifical limitations on the types of the related biological products, and the types of biological products known in the art will do, such as therapeutic products or diagnostic products.

In the following, the attenuated N. seriolae and its construction method and application thereof provided by the present disclosure will be described in detail with reference to the examples, but they should not be understood as limiting the protection scope of the present disclosure.

### Example 1

Method for constructing the attenuated Nocardia seriolae (N. seriolae)

Step 1: Construction of the Homologous Recombinant Vector

Specifically, the preserved wild strain of N. seriolae (ZJ0503) was strak-inoculated into a brain heart infusion (BHI) solid medium (purchased from Guangdong Huankai Microbial Science and Technology Co., Ltd.) under aseptic manipulation, and inverted cultured at 26-30° C. Once single colonies were grown on the plate, a single colony was aseptically picked into 40-60 mL BHI liquid medium, and cultured to logarithmic growth phase at 26-30° C. and 100-150 rpm. 200 μL of bacterial liquid was taken to extract the bacterial genome and use it as a template. The upstream and downstream fragments of the GluNS gene were cloned by using the primers in Table 1, and the fragments were then ligated to the pRE112 vector. The amplification program was: 94° C., 5 min; 30 cycles: 94° C., 30 s, 55° C., 30 s, 72° C., 1 min; and 72° C., 5 min. The amplification system was: 1 μL of genomic DNA of N. seriolae, 2 μL of upstream primer (F) and downstream primer (R), 25 μL of rTaq enzyme, and 20 μL of sterile water.

TABLE 1
Primer sequences

| Primer | | |
|---|---|---|
| name | 5′-3′ sequence | Function |
| GluNS-GF | CATATGGGGCATCACCAGCGGTTCG | To amplify the |
| | (SEQ ID NO: 5) | 389 bp fragment |
| GluNS-GR | [symbol] AG | upstream of the |
| | GAGCATGGCCCCGACT | GluNS gene |
| | (SEQ ID NO: 6) | |
| GluNS-DF | [symbol] CTGACCT | To amplify the |
| | CACTAATTCGGATGCA | 407 bp fragment |
| | (SEQ ID NO: 8) | downstream of |
| GluNS-DR | GAGCTCAATTCGCCGGGTAGTCGC | the GluNS gene |
| | (SEQ ID NO: 9) | |

NOTE:
The sequence marked with underline is the restriction site, and the sequence marked with wavy line is the overlapping PCR site

The E. coli S17-1 strain (purchased from Wuhan Miaoling Biotechnology Co., Ltd.) containing pRE112 plasmid was taken out from the −80° C. refrigerator, melted in a 37° C. water bath, and the melted strains were streaked on a chloramphenicol-resistant LB plate. A single colony was picked for amplification culture overnight, and the plasmid was extracted using a plasmid extraction kit. The pRE112 plasmid and overlapping PCR products were subjected to double enzyme digestion experiments according to the system in Table 2, and the reaction was carried out at 37° C. for 30 min. Then according to Table 3, the overlapping PCR products were ligated with plasmid pRE112. The overlapping PCR products were obtained by the following procedure: overlapping PCR products were obtained by PCR amplification, with GluNS-UF and GluNS-DR as primers, and upstream and downstream fragment of the GluNS gene as templates. The PCR amplification program was: 94° C., 5 min, 15 cycles: 94° C., 30 s; 58° C., 30 s, 72° C., 1 min; and 72° C., 5 min. The reaction system was: GluNS gene upstream fragment product 2 μL, GluNS gene downstream fragment product 2 μL, GluNS-UF and GluNS-DR each 1 μL, rTaq enzyme 25 μL, and sterile water 19 μL.

TABLE 2
Reaction system of the double enzyme digestion

| Composition | Volume |
|---|---|
| DNA | 10 μL |
| Mlu I | 1 μL |
| Xba I | 1 μL |
| ddH₂O | 8 μL |
| Total | 20 μL |

TABLE 3
Ligation system between overlapping PCR and plasmid pRE112

| Composition | Volume |
|---|---|
| Overlapping PCR product | 7 μL |
| Plasmid pRE112 | 1 μL |
| T4 DNA ligase | 1 μL |
| Ligase buffer | 1 μL |
| Total | 10 μL |

Step 2: Preparation of N. seriolae Competent Cells

The preserved wild strain of N. seriolae (ZJ0503) was streak-inculated onto Brain Heart Infusion (BHI) solid medium (purchased from Guangdong Huankai Microbial Science and Technology Co., Ltd.) under aseptic manipulations, and inverted cultured at 28° C. Once single colonies were grown on the plate, a single colony was aseptically picked into 50 mL BHI liquid culture medium and cultured to the logarithmic growth phase at 28° C. and 120 rpm. 30 mL of N. seriolae solution that reached the logarithmic growth phase was taken to a 50 mL centrifuge tube, and the bacterial cells were collected by centrifuge at 4° C., 8000 rpm; the cells were washed twice with 10 mL of 10% sterile glycerol, and the bacterial cells were then resuspended with 10 mL of sterile glycerol.

Step 3: Electroporation

1 μg of knockout plasmid pRE112-ΔGluNS was pipetted and added to competent cells so that the total knockout plasmid was 1 μg, and the plasmids and the cells were mixed in ice bath for 30 min. The mixture was then added to a 96-well microtiter plate, with 100 μL per well. The electroporation parameters were set as follows: voltage 200 V, frequency 30, interval time 1000 ms, and duration 60 ms. After electroporation, 100 μL of BHI liquid medium preheated at 28° C. was added. The mixture were then placed in a 28° C. incubator for 2 hours to recover.

Step 4: Screening of Positive Clones

100 μL of N. seriolae solution recovered after electroporation was spread on a chloramphenicol-resistant BHI plate (25 mg/mL). N. seriolae solution without electroporation, used as a negative control, was spread on a chloramphenicol-resistant BHI plate with an equivalent amount. The plate was placed in a biochemical incubator and inverted cultured at 28° C. until the colonies grew on the plate. A single colony was picked and cultured in BHI liquid medium without 10% sucrose for 5 days. The bacteria was detected whether contained the knockout plasmid pRE112-ΔGluNS by using the primer pairs 112-F1/112-R1 in Table 4. The PCR amplification reaction program was: 94° C., 5 min, 30 cycles: 94° C., 30 s, 55° C., 30 s, 72° C., 1 min, and 72° C., 5 min. The amplification system was: template DNA 1 μL, 112-F1 and 112-R1 each 1 μL, rTaq enzyme 12 μL, and sterile water 10 μL. The bacterial solution was inoculated into BHI liquid medium containing 10% sucrose for continuous culture. Multiplex PCR detection was performed with the primers in Table 5 for deleting strain identification to screen the GluNS gene deletion strain. The PCR amplification reaction program was: 94° C., 5 min, 30 cycles: 94° C., 30 s, (55° C. (GluNS-F2/GluNS-R2), 58° C. (GluNS-F1/GluNS-R1)), 30 s, 72° C., 1 min 72° C., 5 min. The amplification system was: template DNA 1 μL, F and R each 1 μL, rTaq enzyme 12 μL, and sterile water 10 μL.

The 463 bp fragment of the GluNS gene from the wild strain of N. seriolae (ZJ0503) was amplified by using the GluNS-F1/GluNS-R1 primer pair, while the target band could not be amplified from the deletion strain ZJ0503-6296. The GluNS gene of the wild strain of N. seriolae (ZJ0503) and a total of 2042 bp band in the upstream and downstream homologous arms could be amplified by using the GluNS-F2/GluNS-R2 primer pair; but for the deletion strain ZJ0503-6296, only a 796 bp band of upstream and downstream homology could be amplified.

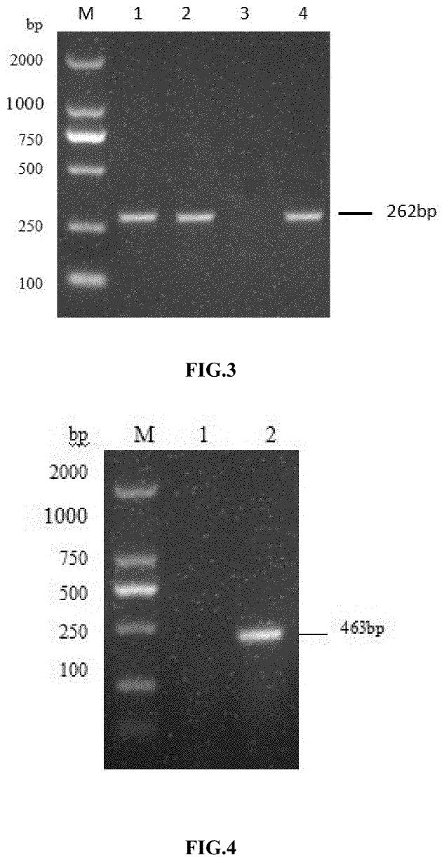

The results are shown in FIG. 3 and FIG. 4. The successfully constructed GluNS gene deletion strain was named as N. seriolae (Nocardia sp.) ZJ0503-6296.

TABLE d
Primers for pRE112 identification

| Primer | | |
|---|---|---|
| name | 5′-3′sequence | Function |
| 112-F1 | ATCTTGCGAATATATGTGTAGA | To identify the |
| | (SEQ ID NO: 2) | presence of pRE112 |
| 112-R1 | TAACCAGACCGTTCAGCTG | plasmid in bacteria |
| | (SEQ ID NO: 3) | |

TABLE 5
Primers for GluNS deletion strain screening

| Primer name | 5′-3′sequence | Function |
|---|---|---|
| GluNS-F1 | CCGCCAAGGACGTGAG | To identify |
| | (SEQ ID NO: 10) | deletion |
| GluNS-R1 | CTGTCACCGATGAGCTGGTA | strains |
| | (SEQ ID NO: 11) | |
| GluNS-F2 | GGGCATCACCAGCGGTTCG | To identify |
| | (SEQ ID NO: 14) | deletion |
| GluNS-R2 | AATTCGCCGGGTAGTCGC | strains |
| | (SEQ ID NO: 15) | |

### Example 2

Genetic Stability Analysis

In order to do determine the genetic stability of the N. seriolae GluNS deletion strain, the N. seriolae ZJ0503-6296 and the wild strain of N. seriolae (ZJ0503) were picked from the plate to a EP tube containing BHI liquid culture medium and were cultured for 3 to 4 days. The cultured strains were then continued to be streak-inoculated on the non-resistant BHI plate and cultured upside down at 28° C. until colonies grew on the plate, during which the colony morphology and growth characteristics were observed. The GluNS deletion strain was continuously streak-inoculated and cultured to 30 generations, and the genetic stability of the corresponding strains was identified with the primers for the deletion strains (GluNS-F2 and GluNS-R2).

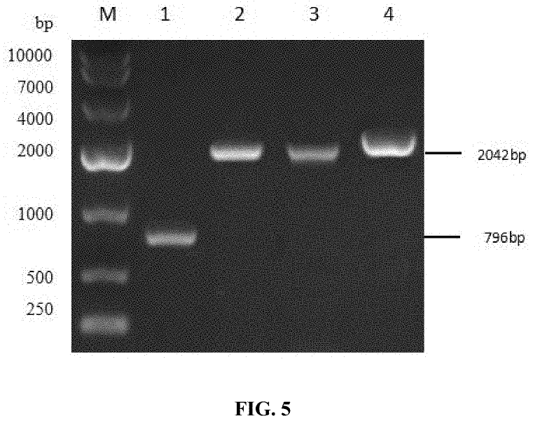

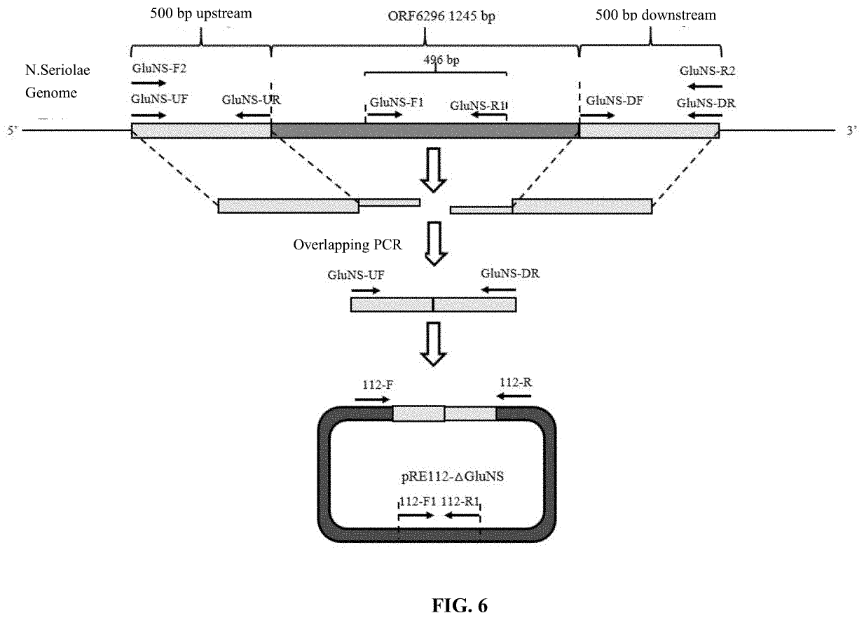

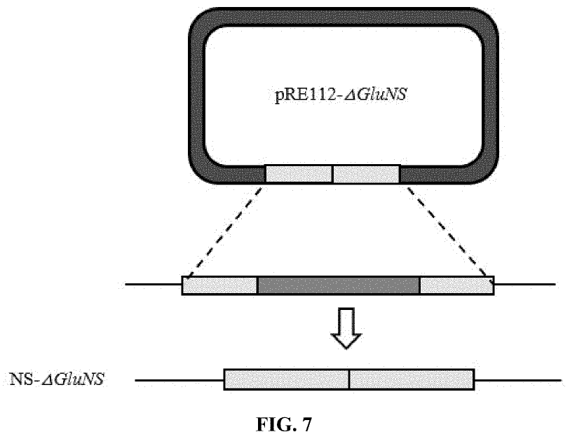

The results are shown in FIG. 5 that the GluNS gene fragment could not be detected when the GluNS deletion strain was passed continuously to 30 generations, indicating that the GluNS deletion strain could be inherited stably.

### Example 3

1. Determination of Median Lethal Concentration

In order to determine the median lethal concentration (LC50) of the deleted strain of N. seriolae ZJ0503-6296 and the wild strain of N. seriolae (ZJ0503). According to the method in the reference^{[2]}, the concentration of the bacterial suspension was adjusted to 10⁴, 10⁵, 10⁶, 10⁷, and 10⁸ CFU/ml with sterile PBS solution, respectively. Each hybrid snakehead in the experimental group was injected intraperitoneally with 100 μL of bacterial solution, and in the control group, each fish was injected with equivalent amount of terile PBS solution, three parallel groups were established for each concentration with 30 fish in each group. Observation was continued for 14 days, commercial feed was fed daily, and dead fish were recorded. According to the method in the reference^{[3]}, statistical analysis was performed and LC₅₀ was calculated by SPSS17.0.

The results are shown in Table 6 and Table 7. The median lethal concentration of the wild strain was 4.74×10⁵ CFU/mL. The median lethal concentration of N. seriolae ZJ0503-6296 was 3.41×10⁶ CFU/mL, which was one order of magnitude lower than that of the wild strain, indicating that the virulence of this strain decreased significantly.

TABLE 6
Statistics on death number of N. seriolae

| | Concentration | | | | |
|---|---|---|---|---|---|
| | 10⁴ | 10⁵ | 10⁶ | 10⁷ | 10⁸ |
| Strain | Death number | | | | |
| N. seriolae ZJ0503-6296 | 0 | 1 | 3 | 7 | 10 |
| Wild strain of N. seriolae (ZJ0503) | 2 | 3 | 5 | 9 | 10 |

TABLE 7
Confidence calculated based on the median lethal concentration
of the deletion strain of N. seriolae ZJ0503-6296

| | | 95% confidence interval | | | 95% confidence interval | | |
|---|---|---|---|---|---|---|---|
| | | of inoculum concentration | | | of log inoculum concentration^{a} | | |
| | | | Lower | Upper | | Lower | Upper |
| | Probability | Estimation | limit | limit | Estimation | limit | limit |
| PRO | 0.010 | 29126.285 | 391.847 | 159506.399 | 4.464 | 2.593 | 5.203 |
| BIT | 0.020 | 50908.468 | 1090.470 | 239896.008 | 4.707 | 3.038 | 5.380 |
| | 0.030 | 72552.376 | 2079.758 | 311960.299 | 4.861 | 3.318 | 5.494 |
| | 0.040 | 94709.978 | 3372.041 | 381037.491 | 4.976 | 3.528 | 5.581 |
| | 0.050 | 117637.186 | 4986.720 | 449187.412 | 5.071 | 3.698 | 5.652 |
| | 0.060 | 141475.137 | 6946.866 | 517496.700 | 5.151 | 3.842 | 5.714 |
| | 0.070 | 166319.540 | 9278.115 | 586650.868 | 5.221 | 3.967 | 5.768 |
| | 0.080 | 192245.218 | 12008.207 | 657139.836 | 5.284 | 4.079 | 5.818 |
| | 0.090 | 219316.720 | 15166.786 | 729348.541 | 5.341 | 4.181 | 5.863 |
| | 0.100 | 247593.559 | 18785.302 | 803602.716 | 5.394 | 4.274 | 5.905 |
| | 0.150 | 409069.672 | 45001.392 | 1215339.396 | 5.612 | 4.653 | 6.085 |
| | 0.200 | 609681.225 | 88458.274 | 1719932.387 | 5.785 | 4.947 | 6.236 |
| | 0.250 | 858585.484 | 155139.370 | 2358958.064 | 5.934 | 5.191 | 6.373 |
| | 0.300 | 1167625.104 | 252244.293 | 3191122.386 | 6.067 | 5.402 | 6.504 |
| | 0.350 | 1552492.436 | 388227.246 | 4304192.363 | 6.191 | 5.589 | 6.634 |
| | 0.400 | 2034386.351 | 572878.187 | 5833368.257 | 6.308 | 5.758 | 6.766 |
| | 0.450 | 2642549.339 | 817609.700 | 7992092.379 | 6.422 | 5.913 | 6.903 |
| | 0.500 | 3418313.219 | 1136220.873 | 1.113E7 | 6.534 | 6.055 | 7.046 |

2. Calculation of Immune Protection Rate

After immunizing the hybrid snakehead with the deletion strain of N. seriolae ZJ0503-6296 at 10⁶ CFU/mL for 35 days, a live bacterial challenge experiment of N. seriolae was performed, and the immune protection rate was calculated. At the same time, a control group in which N. seriolae ZJ0503-6296 was replaced by PBS was set. The immune protection rate was calculated according to formula I.

Relative immune protection rate(RPS)={1−[mortality of immunized group (%)/mortality of control group (%)]}×100% Formula I.

TABLE 8
Calculation results of survival rate and immune protection
rate of hybrid snakehead injected with N. seriolae

| | | Relative immune |
|---|---|---|
| Immunization group | Survival rate | protection rate |
| N. seriolae ZJ0503-6296 | 96% | 93.38% |
| | 98% | |
| | 95% | |
| PBS | 42% | — |
| | 46% | |
| | 46% | |

It could be seen from the above that the relative immune protection rate of the deletion strain N. seriolae ZJ0503-6296 reached 93.38%, which achieved ideal immunogenicity, and the deletion strain N. seriolae ZJ0503-6296 may be used for subsequent vaccine preparation.

### REFERENCES

[1] ZHU Zhidong, Lv Li, Deng Jianhao, et al. Research review of fish nocardiosis[J]. Journal of Aquaculture, 2018, 39(01): 48-52.
[2] Wang Wenji, Chen Jianlin, Hou Suying, et al. A histopathological study of Nocardia seriolae infection in hybrid snakehead[J]. Genomics and Applied Biology, 2019, 38(10): 4439-4446.
[3] Xiong Haoming, Wei Baiqing, Wei Rongjie, et al. Calculation of median lethal dose (LD₅₀) for Yersinia pestis by SPSS package [J]. Chinese Journal of Zoonoses, 2013, 29(11): 1127-1130.

The description of the above embodiments is only used to help understand the method and the core idea of the present disclosure. It should be pointed out that for those of ordinary skill in the art, without departing from the principle of the present disclosure, several improvements and modifications can be made to the present disclosure, and these improvements and modifications also fall within the protection scope of the claims of the present disclosure. Various modifications to these embodiments are obvious to those skilled in the art, and the general principles defined herein can be implemented in other embodiments without departing from the spirit or scope of the present disclosure. Therefore, the present disclosure will not be limited to the embodiments shown herein, but should conform to the widest scope consistent with the principles and novel features disclosed herein.

1. An attenuated Nocardia seriolae, the GluNS gene is deleted in the attenuated Nocardia seriolae.

2. The attenuated Nocardia seriolae according to claim 1, wherein the amino acid of the protein encoded by the GluNS gene is set forth in SEQ ID NO: 12 or a sequence that has a homology or coverage of 30% to 99% with SEQ ID NO:12.

3. The attenuated Nocardia seriolae according to claim 1, wherein the attenuated Nocardia seriolae is deposited with an accession number of GDMCC No: 61258.

4. A method for constructing the attenuated Nocardia seriolaes according to claim 1, comprising the following steps:
1) inserting a homologous recombinant arm of the GluNS gene into a vector to construct a recombinant vector;
2) transforming the recombinant vector into Nocardia seriolae competent cells to obtain recombinant cells;
3) culturing and screening the recombinant cells to obtain an attenuated Nocardia seriolae.

5. The method for constructing the attenuated Nocardia seriolae according to claim 4, wherein the homologous recombinant arm of the GluNS gene in step 1) comprises an upstream fragment and a downstream fragment of the GluNS gene.

6. The method for constructing the attenuated Nocardia seriolae according to claim 5, wherein the length of the upstream fragment or downstream fragment of the GluNS gene is independently 5 bp to 2000 bp.

7. The method for constructing the attenuated Nocardia seriolae according to claim 6, wherein the upstream fragment of the GluNS gene has a 389 bp fragment upstream of the GluNS gene as the upstream homology arm, the downstream fragment has a 407 bp fragment downstream of the GluNS gene as the downstream homology arm, and the homologous recombinant arm of the GluNS gene is formed in a manner of ligating the upstream homology arm-the downstream homology arm.

8. The method for constructing the attenuated Nocardia seriolae according to claim 7, wherein the nucleotide sequence of the homologous recombinant arm of the GluNS gene in step 1) is set forth in SEQ ID NO:1.

9. The method for constructing the attenuated Nocardia seriolae according to claim 4, wherein the vectors in step 1) comprise pRE112; the multiple cloning site where the homologous recombinant arm of the GluNS gene is inserted into pRE112 is Sac I/Kpn I.

10. The method for constructing the attenuated Nocardia seriolae according to claim 4, wherein the culture solution in step 3) is BHI solution containing 0.3 mM sucrose preheated at 26-30° C., the culture temperature is 26-30° C., and the culture time is 10-12 h.

11. The method for constructing the attenuated Nocardia seriolae according to claim 4, wherein the transformation in step 2) comprises electroporation; the parameters of the electroporation is as follows: voltage 180-220 V, pulse interval time 800-1200 ms, pulse duration 80-120 μs, and square wave 25-35.

12. The method for constructing the attenuated Nocardia seriolae according to claim 4, wherein the screening comprises detecting whether the bacteria contains the knockout plasmid pRE112-ΔGluNS by a PCR amplification method;
the primer pair for PCR amplification is 112-F1/112-R1;
the nucleotide sequence of 112-F1 is set forth in SEQ ID NO: 2;
the nucleotide sequence of 112-R1 is set forth in SEQ ID NO: 3;
clones in which the GluNS gene fragment is shown to be deleted via a PCR amplification product sequencing, and the bacteria contains a knockout plasmid pRE112-ΔGluNS are positive clones.

13. The method for constructing the attenuated Nocardia seriolae according to claim 4, wherein after the screening, the method further comprises an identification of the attenuated Nocardia seriolae;
the identification comprises identifying whether the GluNS gene is deleted by a PCR amplification method;
the primer pairs for PCR amplification are GluNS-F1/GluNS-R1 and GluNS-F2/GluNS-R2;
the nucleotide sequence of GluNS-F1 is set forth in SEQ ID NO: 10;
the nucleotide sequence of GluNS-R1 is set forth in SEQ ID NO: 11;
the nucleotide sequence of GluNS-F2 is set forth in SEQ ID NO: 14;
the nucleotide sequence of GluNS-R2 is set forth in SEQ ID NO: 15;
the target band is not amplified from GluNS gene deletion strains by using the GluNS-F1/GluNS-R1 primer pair; only a 796 bp upstream and downstream homologous band is amplified from GluNS gene deletion strains by using the GluNS-F2/GluNS-R2 primer pair.

14. A method of preventing Nocardiosis by administering a vaccine or a related biological product, wherein the vaccine or related biological product comprises the attenuated Nocardia seriolae according to claim 1.

15. A vaccine or related biological products, wherein the vaccine or related biological products comprises the attenuated Nocardia seriolae according to claim 1 as an active component.

16. The attenuated Nocardia seriolae according to claim 3, wherein the amino acid of the protein encoded by the GluNS gene is set forth in SEQ ID NO: 12 or a sequence that has a homology or coverage of 30% to 99% with SEQ ID NO:12,

17. The method for constructing the attenuated Nocardia seriolae according to claim 13, wherein the homologous recombinant arm of the GluNS gene in step 1) comprises an upstream fragment and a downstream fragment of the GluNS gene.

18. The method for constructing the attenuated Nocardia seriolae according to claim 13, wherein the length of the upstream fragment or downstream fragment of the GluNS gene is independently 5 bp to 2000 bp.

19. The method for constructing the attenuated Nocardia seriolae according to claim 13, wherein the upstream fragment of the GluNS gene has a 389 bp fragment upstream of the GluNS gene as the upstream homology arm, the downstream fragment has a 407 bp fragment downstream of the GluNS gene as the downstream homology arm, and the homologous recombinant arm of the GluNS gene is formed in a manner of ligating the upstream homology arm-the downstream homology arm.

20. The method for preventing Nocardiosis according to claim 14, wherein the amino acid of the protein encoded by the GluNS gene is set forth in SEQ ID NO: 12 or a sequence that has a homology or coverage of 30% to 99% with SEQ ID NO:12.


SEQUENCE LISTING (15 sequences)

SEQ ID NO: 1 - DNA, 1035 bp, Artificial Sequence
  DNA sequence of the recombination homology arm of the GluNS gene
gcctgcaccctgggcggtgacagcggcggcgcgatcgtgtcgggcacgctggcgctgggcatcaccagcggttcgaacgcggccgacgcgccgaactgcaacgaggccaacaccgcgctggcgcagtacggtggaaccgcgtcgctgggcatcccggtgcgcgcgattctcaccgaaatcgacgccaattccggtggcggcgtcggcagcggaattcaggtgcggacgcggtccaacgcctgacggcgaagtgctccagcacccgacggcaaagcggtccaacgcctgacgatggcgacatcggcggtaaacggacatccttcacattgcccctgaatccgacatgcccgctgtttgccccgtgaataccgaaacgtcacctcgcaataagtaggcataacgggcccgacaccatatagtcggggccatgctcctgccacgtatgcgcttctccatgcctgccgcgggccgacgcccgcggtcccttgtccgtactgcggcgatcgcggcgacctcttcacggcaccctcgaaaaggccggtagctcaacagctaccggccttttgtcgtcccggtggggaagagataccgctgacctcactaattcggatgcagcgctggaagttcgtggcccgcctcggtctggactgacggagcgggggagcgaagcggaggagcggaggagggaagaccgaggtaacagggccaccaaccgccgtagcgcagcggaggcaaattaaacagagcatgctggggatatgtgtttggtgttgctgggttggcgagcgcatccggagtaccgcttgatcgtggccgccaaccgggacgagttcttcacccgcccaacggaatcgctgcgccggtgggacgaagtgcccggggtgctggccgggcgggacctgggcgcggccggaccggtgcccggcacctggctcggtgcgctgcccgatcatcgccgcttcgcgacggtcaccaatgtgcgactacccggcgaattccgcgccgatgtgcgctcgcgcggcgcgctgctgctgg

SEQ ID NO: 2 - DNA, 22 bp, Artificial Sequence
  Primer 112-F1
atcttgcgaatatatgtgtaga

SEQ ID NO: 3 - DNA, 19 bp, Artificial Sequence
  Primer 112-R1
taaccagaccgttcagctg

SEQ ID NO: 4 - DNA, 517 bp, Artificial Sequence
  DNA sequence of the upstream fragment of the GluNS gene
gcctgcaccctgggcggtgacagcggcggcgcgatcgtgtcgggcacgctggcgctgggcatcaccagcggttcgaacgcggccgacgcgccgaactgcaacgaggccaacaccgcgctggcgcagtacggtggaaccgcgtcgctgggcatcccggtgcgcgcgattctcaccgaaatcgacgccaattccggtggcggcgtcggcagcggaattcaggtgcggacgcggtccaacgcctgacggcgaagtgctccagcacccgacggcaaagcggtccaacgcctgacgatggcgacatcggcggtaaacggacatccttcacattgcccctgaatccgacatgcccgctgtttgccccgtgaataccgaaacgtcacctcgcaataagtaggcataacgggcccgacaccatatagtcggggccatgctcctgccacgtatgcgcttctccatgcctgccgcgggccgacgcccgcggtcccttgtccgtactgcggcgatcgcggcgacctct

SEQ ID NO: 5 - DNA, 25 bp, Artificial Sequence
  Primer GluNS-UF
catatggggcatcaccagcggttcg

SEQ ID NO: 6 - DNA, 41 bp, Artificial Sequence
  Primer GluNS-UR
tgcatccgaattagtgaggtcagaggagcatggccccgact

SEQ ID NO: 7 - DNA, 518 bp, Artificial Sequence
  DNA sequence of the downstream fragment of the GluNS gene
tcacggcaccctcgaaaaggccggtagctcaacagctaccggccttttgtcgtcccggtggggaagagataccgctgacctcactaattcggatgcagcgctggaagttcgtggcccgcctcggtctggactgacggagcgggggagcgaagcggaggagcggaggagggaagaccgaggtaacagggccaccaaccgccgtagcgcagcggaggcaaattaaacagagcatgctggggatatgtgtttggtgttgctgggttggcgagcgcatccggagtaccgcttgatcgtggccgccaaccgggacgagttcttcacccgcccaacggaatcgctgcgccggtgggacgaagtgcccggggtgctggccgggcgggacctgggcgcggccggaccggtgcccggcacctggctcggtgcgctgcccgatcatcgccgcttcgcgacggtcaccaatgtgcgactacccggcgaattccgcgccgatgtgcgctcgcgcggcgcgctgctgctgg

SEQ ID NO: 8 - DNA, 41 bp, Artificial Sequence
  Primer GluNS-DF
agtcggggccatgctcctctgacctcactaattcggatgca

SEQ ID NO: 9 - DNA, 24 bp, Artificial Sequence
  Primer GluNS-DR
gagctcaattcgccgggtagtcgc

SEQ ID NO: 10 - DNA, 16 bp, Artificial Sequence
  Primer GluNS-F1
ccgccaaggacgtgag

SEQ ID NO: 11 - DNA, 20 bp, Artificial Sequence
  Primer GluNS-R1
ctgtcaccgatgagctggta

SEQ ID NO: 12 - protein, 414 aa, Artificial Sequence
  Amino acid sequence of the protein encoded by the GluNS gene
VLVAGPLAASAHAEPATPDLPAQLIAAITRDLKISPQDYLARADTAQKVATFATTAQRQFPQVFGGAWLDETGKAVVALAPGEGVDKARKAVQDAGFTAKDVSKSETTLRGEKNAFQQWLKDQPESVAKAIRGVAIDTLNNSIAVRVDKPDLPLPGFVDPARVIVMTAPPVGGEGQNVPQATEIAGAGPRAIAAGEAYASVAGRMSLRCSLGFNGTDGNGNVVNITAGHCNPNIAATGGANSPSVYQLIGDSRGPEVGQFQKSVLGNEDYSIVGINDQFRDAFSNPFVTVPGSASIAVTGVAVPVVGAPVCKSGARTGFSCGVVNAVDQTVQVGDRLLTQSFSANICALPGDSGGPLVTGTLALGIASASSVADYPICEIPNLLGLITGNTPQLFAQPVSTVLSDNPGLRVRTT

SEQ ID NO: 13 - DNA, 1245 bp, Artificial Sequence
  DNA sequence of the GluNS gene
gtgctcgtcgccggacccctggcggcgagcgcacacgcggaacccgcgacccccgatctgcccgcccagctgatcgcggccatcacccgcgacctcaagatctccccgcaggactacctggcccgggccgacaccgcacagaaggtggccaccttcgccaccaccgcgcagcggcagttcccgcaggtcttcggcggcgcctggctggacgagaccggcaaggccgtcgtggcgctggccccgggcgagggcgtggacaaggcccgcaaggccgtgcaggacgccggtttcaccgccaaggacgtgagcaagagcgagaccacgctgcgcggcgagaagaacgccttccagcagtggctcaaggatcagcccgagtccgtggcgaaggccatccggggcgtggccatcgacaccttgaacaacagcatcgcggtgcgggtggacaagcccgatctgccgctgccgggcttcgtggacccggcccgcgtcatcgtcatgaccgcaccgccggtcggcggcgagggccagaacgtgccgcaggccaccgagatcgcgggcgcgggcccgcgcgccatcgccgccggcgaggcctacgcctcggtcgccggccgcatgtcgctgcgctgctcgctcggcttcaacggcaccgacggcaacggcaatgtcgtcaatatcaccgcgggccactgcaacccgaacatcgcggccaccggcggcgcgaactcgccgagcgtgtaccagctcatcggtgacagccgcggtcccgaggtcggccagttccagaagtcggtgctgggcaacgaggactactcgatcgtcggcatcaacgaccagttccgcgacgccttctccaacccgttcgtaaccgtcccgggctcggcctcgatcgccgtcaccggcgtggccgtgccggtggtcggcgcgccggtctgcaagtcgggtgcgcgcaccggcttcagctgcggcgtggtgaacgccgtggaccagaccgtccaggtcggcgaccgcctgctgacccagtccttctccgccaatatctgtgccctgcccggtgattcgggtggcccgctggtgaccggcacgctggccctgggcatcgccagcgcatcctcggtcgccgactacccgatctgcgagatcccgaacctgctcggcctgatcaccggcaacaccccgcagctgttcgcgcagccggtgagcaccgtgctctccgacaacccggggctgcgggtccgcaccacgtaa

SEQ ID NO: 14 - DNA, 19 bp, Artificial Sequence
  Primer GluNS-F2
gggcatcaccagcggttcg

SEQ ID NO: 15 - DNA, 18 bp, Artificial Sequence
  Primer GluNS-R2
aattcgccgggtagtcgc